(19) United States
(12) Patent Application Publication
(10) Pub. No.: US 2019/0060415 A1
(43) Pub. Date: Feb. 28, 2019

(54) HEALTH PROMOTING COMPOSITION AND METHODS OF USE SUPPLEMENT

(71) Applicant: Lonza Inc., Allendale, NJ (US)

(72) Inventor: Aouatef Bellamine, Pennington, NJ (US)

(21) Appl. No.: 16/118,645

(22) Filed: Aug. 31, 2018

Related U.S. Application Data
(60) Provisional application No. 62/552,470, filed on Aug. 31, 2017.

(51) Int. Cl. A61K 38/39; A61K 9/00; A61K 31/455; A61K 31/405; A61K 31/7084; A61K 38/17; A61K 31/19; A61K 35/741

(52) U.S. Cl. CPC A61K 38/39; A61K 9/0053; A61K 9/0014; A61K 31/455; A61K 2035/115; A61K 31/7084; A61K 38/177; A61K 31/19; A61K 35/741; A61K 31/405

(57) ABSTRACT

The present disclosure is directed to a composition or nutritional supplement containing undenatured Type II collagen in combination with a Vitamin B compound or derivative thereof or any agent that can bind and/or activate the GPR109a receptor such as butyrates and dietary fibers and probiotics resulting to an increase in butyrates. The different dietary or nutraceutical agents are capable of synergistically working together to improve the health and well-being of a human or animal. For instance, the nutritional supplement can be used to treat joint discomfort and pain, improve immune, gastrointestinal and brain health, lowering cholesterol, or otherwise reducing inflammation in human and animal populations both at risk and actively presenting with such ailments.

### RELATED APPLICATIONS

The present application is based on and claims priority to U.S. Provisional Patent Application Ser. No. 62/552,470 having a filing date of Aug. 31, 2017, which is incorporated herein by reference in its entirety.

### BACKGROUND

In recent years, the use of collagen to treat various conditions has become exceedingly popular. Collagen is a protein that can be found in muscles, bones, skin, blood vessels, and in other parts of the body. There are various different types of collagen depending upon its function and form. For instance, Type I collagen, the most abundant collagen, is made of fibers found in tendons, ligaments, organs and skin. Type II collagen, on the other hand, primarily helps build cartilage, a major structural entity that sits on the surfaces of those bones which comprise articulating joints. Type III collagen, on the other hand, is made of fibers and is a major component of the extra cellular matrix that makes up organs and skin. Type III collagen also forms blood vessels and tissue within the heart.

Collagen can be found in numerous different products including cosmetic creams and body lotions. Collagen is also found in various oral supplements. Collagen production in the body, for instance, tends to slow as a person ages. Thus, collagen supplements have been taken in the past in order to reduce the effects of aging by improving the health of skin and hair.

Collagen has also been found to effectively treat arthritis and other joint pain. For example, U.S. Pat. No. 9,066,926 discloses a method of reducing exercise-induced joint pain in mammals by administering Type II collagen to a mammal. The '926 patent is incorporated herein by reference.

Various other supplements have been proposed containing bone material. For instance, U.S. Pat. No. 9,011,930 is directed to nutritional supplements containing demineralized bone matrix. The supplement may further contain a vitamin and is used to maintain or improve the condition of bones or cartilage.

In addition to treating joint pain, collagen is also known to treat leaky gut syndrome, a condition where toxins are able to pass through a digestive tract. Collagen intake can also boost metabolism, muscle mass, and energy output. Collagen can also improve liver health and improve cardiovascular health.

Although collagen can offer various advantages when administered to a human or animal, a need exists for a composition and method that can increase the effectiveness of collagen and/or work in conjunction with collagen to provide synergistic effects.

### SUMMARY

In general, the present disclosure is directed to a composition containing the combination of a collagen source and a Vitamin B compound or derivative, or precursor thereof, or a GPR109a receptor ligand or any compound that results in increased butyrate levels. The composition of the present disclosure has numerous and diverse applications and uses. In one embodiment, for instance, the present disclosure is directed to a nutritional supplement. For example, in one embodiment, the nutritional supplement of the present disclosure contains Type II collagen combined with a Vitamin B compound or derivative, or precursor thereof or a GPR109a ligand. The collagen source, for instance, may comprise denatured collagen or undenatured collagen. In one embodiment, the collagen source comprises hydrolyzed collagen. The collagen can be obtained from various different sources, such as animal sources, and can be supplied as animal parts or can be supplied as a pure protein. The resulting composition can have many different health benefit effects when administered to a human, an animal, and/or mammal. For instance, the composition is well suited to treating or managing any sort of joint pain, discomfort, or limited performance. The composition of the present disclosure is also well suited to reducing inflammation, improving immune system health, lowering cholesterol, and/or reducing the risk of atherosclerosis.

In addition to a nutritional supplement, the composition of the present disclosure can also be used as a medicinal composition in order to deliver various health benefits to a human or animal. The medicinal composition, for instance, can be administered orally, subcutaneously, topically, intravenously, or by any other suitable method.

In still another embodiment of the present disclosure, the composition containing a collagen source combined with a Vitamin B compound, or derivative, or precursor, or a GPR109a receptor ligand, can be contained in a topical formulation that is to be applied to the skin. For instance, the topical composition can be in the form of gel or cream and can contain numerous and different ingredients depending upon the end use application. For instance, the topical composition may contain solvents, surfactants, emulsifiers, consistency factors, conditioners, emollients, skin caring ingredients, moisturizers, thickeners, lubricants, and the like. The topical composition can be applied to the skin of a user for improving skin health. Alternatively, the composition can be applied to the skin for absorption into the body for providing one or more health benefits. Such composition can be given orally to improve skin health.

As described above, the composition of the present disclosure generally comprises a collagen source combined with a Vitamin B compound, or derivative, or precursor thereof, or a GPR109a receptor ligand. The Vitamin B compound, or derivative thereof, can comprise, for instance, a nicotinate. Particular examples of Vitamin B compounds or derivatives, or precursors thereof include niacin, nicotinic acid, niacinamide, nicotinamide, tryptophan, nicotinamide dinucleotide (NAD), or mixtures thereof. GPR109a receptor ligands include Niacin, butyrates, or any agent that can lead to the stimulation of the receptor or the increase in butyrates and nicotinic acids. Examples include dietary fibers and probiotics. In other embodiments, the Vitamin B compound or derivative, or precursor thereof, may comprise a niacinamide N-oxide, a salt of a niacinamide, nicotinamide adenine dinucleotide phosphate, a tryptophan dipeptide, a nicotinyl alcohol, a nicotinic acid ester, a nicotinyl amino acid, a nicotinyl alcohol ester, or mixtures thereof.

In one embodiment, the composition can be a nutritional supplement in the form of individual dosage vessels. Each dosage vessel can be made from an orally acceptable composition. The dosage vessels, for instance, may comprise capsules, tablets, or the like. Alternatively, the nutritional supplement may be in the form of a syrup or liquid suspension. Alternatively, the composition of the present embodiment can be administered in a food or a beverage form.

When in the form of individual dosage vessels, each dosage vessel can contain the Type II collagen source in an amount from about 1 mg to about 15,000 mg, such as from about 1 mg to about 2,000 mg, such as from about 1 mg to about 1,000 mg, such as from about 20 mg to about 100 mg. The Vitamin B compound, or derivative, or precursor thereof, or GPR109a receptor ligands on the other hand, can be present in each individual dosage vessel in an amount from about 0.1 mg to about 10,000 mg, such as from about 0.5 mg to about 10,000 mg, such as from about 1 mg to about 8,000 mg.

The present disclosure is also directed to methods for treating or managing various ailments. For instance, in one embodiment, the present disclosure is directed to a method for treating or managing joint pain, joint discomfort, or limited joint flexibility in individuals who are not diseased (“healthy subjects”), and whose symptoms are due to participating in normal, daily activities including, but not limited to, strenuous exercise, walking, running, and so on. In another embodiment, the present disclosure is also directed to a method for treating joint ailments, as described above, that are due to a disease or age (including arthritis) in humans and other mammals. The method includes administering to the mammal a therapeutically effective amount of the composition or supplement as described above. In one embodiment, the composition contains undenatured Type II collagen in combination with a Vitamin B compound, or derivative, or precursor thereof, or a GPR109a receptor ligand. The composition, which can be in the form of oral dosage vessels, can be administered to the mammals in an amount sufficient to reduce the joint discomfort or pain, or begin and support the healing process, or any combination thereof.

The present disclosure is also directed to various methods to improving the health of a user in addition to reducing joint discomfort or pain. For instance, in one embodiment, the composition of the present disclosure can be used to improve immune system health, and/or decrease inflammation. In addition, the composition of the present disclosure can be used to lower cholesterol in a user, or reduce the risk of atherosclerosis or improve gut and brain health, or any combination thereof.

### Definitions

The term “therapeutically effective amount” as used herein, with respect to the composition described above, shall mean that dosage, or amount of composition, that provides the specific pharmacological or nutritional response for which the composition (containing the collagen source combined with a Vitamin B compound, or derivative, or precursor thereof, or GPR109a receptor ligand) is administered or delivered to subjects in need of such treatment. It is emphasized that “therapeutically effective amount”, administered to a particular subject in a particular instance will not always be effective in treating the ailments or otherwise improve health as described herein, even though such dosage is deemed a “therapeutically effective amount” by those skilled in the art. Specific subjects may, in fact, be “refractory” to a “therapeutically effective amount”. For example, a refractory subject may have a low bioavailability, genetic variability in a specific receptor, metabolic pathway or response capacity, such that clinical efficacy is not obtainable. It is to be further understood that the composition, or supplement, in particular instances, can be measured as oral dosages, or with reference to ingredient levels that can be measured in blood. In other embodiments, dosages can be measured in amounts applied to the skin when the composition is contained with a topical formulation.

The term “delivering” or “administering” as used herein, refers to any route for providing the composition, or a nutraceutical, to a subject as accepted as standard by the medical community. For example, the present invention contemplates routes of delivering or administering that include oral ingestion plus any other suitable route of delivery including transdermal, intravenous, intraperitoneal, intramuscular, topical or subcutaneous.

The term “encapsulate”, “encapsulated”, or “encapsulating” refers to any compound that is completely surrounded by a protective material. For example, a compound may become encapsulated by a population of nanoemulsion particle formation during microfluidization.

The term “nutraceutical” refers to any compound added to a dietary source (i.e., for example, a fortified food or a dietary supplement) that provides health or medical benefits in addition to its basic nutritional value.

Other features and aspects of the present disclosure are discussed in greater detail below.

### DETAILED DESCRIPTION

It is to be understood by one of ordinary skill in the art that the present discussion is a description of exemplary embodiments only, and is not intended as limiting the broader aspects of the present disclosure.

In general, the present disclosure is directed to compositions, such as nutritional supplements, medicinal or food formulations, or topical compositions for administering to humans and animals that can be used as a daily supplement or can be used to treat a particular ailment. Of particular advantage, the composition can be formulated so as to treat or improve the health of many different organ systems within the body. In fact, the composition of the present disclosure can provide multiple health benefits simultaneously.

In one embodiment, for instance, the composition of the present disclosure is particularly formulated to improve joint health. For instance, the composition can be used to treat arthritis or non-arthritic joint pain, joint discomfort, or lack of joint flexibility. The composition of the present disclosure can also be used to improve immune system health, prevent or reduce inflammation, lower cholesterol, and/or lower the risk of atherosclerosis and improve brain and gut health.

In general, the composition of the present disclosure contains a collagen, such as denatured or undenatured Type II collagen in combination with another dietary or nutraceutical agent that, in one embodiment, can work synergistically with the collagen. For example, in one embodiment, the dietary or nutraceutical agent can increase the effectiveness of the collagen. In addition, or alternatively, the combination of collagen with the dietary or nutraceutical agent, or ingredient, can provide a platform for treating more than one aliment or otherwise improving the health of multiple organ systems within the body.

In one embodiment, for instance, Type II collagen is combined with a Vitamin B compound, or derivative, or precursor thereof, or a GPR 109a receptor ligand. The composition or nutritional supplement formulated in accordance with the present disclosure can be administered in an oral form. Other enteral modes of administration are also possible. In one embodiment, the nutritional supplement is formed into a dosage vessel, such as a tablet or capsule that can be taken orally. In addition, the nutritional supplement can be incorporated into beverages, foods and dietary supplements. The nutritional supplement, for instance, can be manufactured in the form of capsules, tablets, gummy chewables, edible films, lozenges, powders, liquid suspensions, syrups, and the like. In one embodiment, the nutritional supplement is encapsulated by a protective material in forming a capsule or tablet. Alternatively, the composition can be administered subcutaneously, intravenously, or can be applied topically to the skin of a user.

The composition can be taken regularly or can be taken for a temporary period of time in order to treat or manage a particular condition. As indicated above, the composition contains a collagen, particularly a Type II collagen, such as an undenatured Type II collagen. Type II collagen for use in the present disclosure can be obtained from any suitable source. For instance, the collagen can be derived from a variety of mammalian sources, avian sources, or can be obtained from various fish species. For instance, the collagen can be obtained from salmon, shark, poultry, egg shells, turkey cartilage, bovine cartilage, and the like. In one embodiment, for instance, the Type II collagen can be obtained as disclosed in U.S. Pat. No. 7,083,820 to Schilling which is incorporated by reference. For example, undenatured Type II collagen is available commercially as the UC-II® brand from InterHealth Nutraceuticals. The UC-II® brand is a natural ingredient that contains a glycosylated, undenatured Type II collagen derived from chicken sternum. The collagen source can also comprise a hydrolyzed collagen as well as a pure protein. In one embodiment, the collagen source can be free of any bone or bone material. In other embodiments, the collagen source can be free of any transforming growth factors-beta, bone morphogenic proteins, or both. In still another embodiment, the collagen source comprises Type II collagen and is completely free of any Type I collagen.

In preparing animal tissue for oral administration, in one embodiment, the Type II collagen containing tissue can be first dissected free of surrounding tissues and diced or otherwise comminuted into particles. The particulate, or milled, cartilage can be sterilized by means which do not affect or denature the structure of a major portion of the Type II collagen in the tissue and formed into doses containing therapeutically effective levels of undenatured Type II collagen, said levels being generally in the amount of at least about 0.01 gram and preferably from about 0.1 to about 0.5 grams of animal tissue in a dose. Being a natural product some variation from sample to sample is to be expected. These variations can be minimized by blending after comminution. The blending can be aided by analytical techniques which allow the measurement of the amount of undenatured Type II collagen and other antigens.

These measurements will allow blending of batches to obtain uniformity and in some cases to modify potency by increasing certain antigen levels by mixing cartilage from different sources. The optimum dosage may vary.

In accordance with the present disclosure, the Type II collagen is combined with a Vitamin B compound, or derivative, or precursor thereof, or a GPR109a receptor ligand. In one embodiment, the Vitamin B compound can comprise a compound having the following formula:

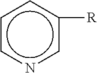

wherein R is —CONH₂ (i.e., niacinamide), —COOH (i.e., nicotinic acid) or —CH₂OH (i.e., nicotinyl alcohol).

The Vitamin B compound or derivative thereof can also comprise any suitable nicotinate. In addition to niacin, niacinamide, and nicotinyl alcohol, for instance, the Vitamin B compound can also comprise nicotinamide, nicotinamide dinucleotide, and the like. As used herein, Vitamin B compounds also include Vitamin precursors that convert to a Vitamin B compound within the body. For instance, tryptophan converts into niacin within the body.

As used herein, a GPR109a receptor ligand is any suitable compound that can couple to the protein GPR109a and also includes compounds that change form within the body, i.e. metabolize, so that they can couple to the GPR109a protein. GPR109a receptor ligands, for instance, include short chain fatty acids. GPR109a receptor ligands also can include dietary fibers, probiotics, or any other suitable precursor which, when administered, converts to a GPR109a receptor, such as a short chain fatty acid. Such short chain fatty acids can include butyric acid and other similar fatty acids. GPR109a receptor ligands can also include salts of such fatty acids, such as butyrates.

Derivatives of Vitamin B compounds include nicotinic acid esters, nicotinyl amino acids, nicotinyl alcohol esters, nicotinic acid N-oxides, niacinamide N-oxides, and the like.

Suitable esters of nicotinic acid include nicotinic acid esters of C1 to C22 alcohols. The alcohol can be straight chain or branched, cyclic or acyclic, saturated or unsaturated, and substituted or unsubstituted.

Exemplary nicotinyl alcohol esters include nicotinyl alcohol esters of the carboxylic acids salicylic acid, acetic acid, glycolic acid, palmitic acid and the like. Other non-limiting examples of vitamin B compounds useful herein are 2-chloronicotinamide, 6-aminonicotinamide, 6-methylnicotinamide, n-methylnicotinamide, n,n-diethylnicotinamide, n-(hydroxymethyl)-nicotinamide, quinolinic acid imide, nicotinanilide, n-benzylnicotinamide, n-ethylnicotinamide, nifenazone, nicotinaldehyde, isonicotinic acid, methyl isonicotinic acid, thionicotinamide, nialamide, 1-(3-pyridylmethyl) urea, 2-mercaptonicotinic acid, nicomol, and niaprazine.

Still other suitable Vitamin B compounds or derivatives, or precursors include nicotinamide adenine dinucleotide phosphate, tryptophan dipeptide, tocopherol nicotinate, and the like.

Vitamin B compounds and derivatives, and precursors thereof also include salts of Vitamin B compounds. Nonlimiting examples of salts of the vitamin B compound useful herein include organic or inorganic salts, such as inorganic salts with anionic inorganic species (e.g., chloride, bromide, iodide, carbonate, preferably chloride), and organic carboxylic acid salts (including mono-, di- and tri-C1-C18 carboxylic acid salts, e.g., acetate, salicylate, glycolate, lactate, malate, citrate, preferably monocarboxylic acid salts such as acetate).

Vitamin B intake in animals and humans can provide numerous and diverse health benefits. For instance, nicotinates generally assist in energy metabolism within the body as they supply NAD and NADP in the oxidation-reduction reactions that occur in synthesizing energy.

Vitamin B compounds, and derivatives thereof, can also lower cholesterol in the body. For example, niacin can lower cholesterol levels and/or prevent vascular diseases, such as atherosclerosis.

In addition, Vitamin B compounds, and derivatives thereof, and precursors, have been found to be well matched with a Type II collagen for reducing joint pain. For instance, Type II collagen can have an effect on T-regulatory cells that produce anti-inflammatory cytokines that switch chondrocyte physiology from matrix proteins breakdown to the creation and deposition of matrix proteins. This process appears to require the active participation of vitamin B3, as knockout mutants of GPR109a, a vitamin B3 receptor, has been shown to significantly decrease the activation of T-regulators. In this manner, the Vitamin B compounds, and derivatives, and precursors thereof, can synergistically operate with the Type II collagen to provide overall better efficacy. Butyrates are believed to affect this pathway by interacting with the same receptor. In addition, GPR109a receptor is expressed on dendritic cells in the gut, at the same site of action where the UC-II brand mediates its effects. Vitamin B compounds on their own can also improve joint mobility while reducing joint discomfort and inflammation. For example, in one embodiment, it is believed that the combination of a Type II collagen and a Vitamin B compound, a derivative, or precursor thereof, or butyrates, can provide an overall better composition for treating joint pain resulting from participation in normal, everyday activities.

Vitamin B compounds, and derivatives thereof, can also improve immune system health. Vitamin B compounds can also generally reduce inflammation within the body. For instance, the niacin/GRP109a receptor interaction leads to the formation of anti-inflammatory prostaglandins. The reduced inflammatory effects can be felt in joints and in other parts of the body.

The amount of Type II collagen and the amount of the Vitamin B compound, or derivative, or precursor thereof, in a dose consumed at any given time will vary with the purpose of the consumption, the severity of symptoms, as well as the condition, age, weight, medical history and general physical characteristics of the subject (human or animal) to be treated. Consequently, the dosages, the frequency, and time period over which the dosages are administered can vary widely. The nutritional supplement of the present disclosure can be combined with other digestible ingredients such as in the form of aqueous dispersions, such as milk, or in combination with other protein-rich substances, sugars and starches. In one embodiment, the nutritional supplement may be administered directly as a comminuted solid as in an encapsulated comminuted solid, such as a compression and formed pill, as well as a slurry with or without other digestible compositions such as, for example, foodstuffs.

In one embodiment, the weight ratio between the Type II collagen and the Vitamin B compound or derivative, or precursor thereof can generally be from about, 15,000:1 to about 1:500, such as from about 400:1 to about 1:800, such as from about 100:1 to about 1:500, such as from about 50:1 to about 1:200, such as from about 10:1 to about 1:150, such as from about 1:1 to about 1:100, such as from about 1:1 to about 1:80, such as from about 1:1 to about 1:50. In one embodiment, for instance, the weight ratio between collagen and the Vitamin B compound or derivative, or precursor thereof is from about 40:1 to about 1:2.5. In an alternative embodiment, the weight ratio between the collagen and the Vitamin B compound or derivative, or precursor thereof is from about 1:5 to about 1:50. In still another embodiment, the weight ratio between collagen and the Vitamin B compound or derivative, or precursor thereof is from about 1:1.5 to about 1:150. In yet another embodiment, the weight ratio between the collagen and the Vitamin B compound or derivative, or precursor thereof is from about 1:1.1 to about 1:4.

In one embodiment, the composition or nutritional supplement of the present disclosure is formed into individual dosage vessels that are intended to be taken orally by a human or animal. The dosage vessel, for instance, may comprise a tablet or capsule. In one embodiment, the Type II collagen can be present in each dosage vessel generally in an amount from about 1 milligram to about 15,000 milligrams. For example, when denatured or hydrolyzed collagen is present in the composition, the collagen can be present in each dosage vessel in an amount from about 500 milligrams to about 15,000 milligrams. When undenatured Type II collagen is used to produce the composition, on the other hand, each dosage vessel can contain the collagen in an amount from about 1 milligram to about 5,000 milligrams. For instance, the Type II collagen can be present in each dosage vessel in an amount greater than about 5 milligrams, such as in an amount greater than about 10 milligrams, such as in an amount greater than about 15 milligrams, such as in an amount greater than about 20 milligrams, such as in an amount greater than about 25 milligrams, such as in an amount greater than about 30 milligrams. The amount of Type II collagen present in each dosage vessel can generally be less than about 1000 milligrams, such as less than about 900 milligrams, such as less than about 800 milligrams, such as less than about 700 milligrams, such as less than about 600 milligrams, such as less than about 500 milligrams, such as less than about 400 milligrams, such as less than about 300 milligrams, such as less than about 200 milligrams, such as less than about 100 milligrams, such as less than about 80 milligrams, such as less than about 70 milligrams, such as less than about 60 milligrams.

The Vitamin B compound or derivative, or precursor thereof can be present in the dosage vessel in an amount generally greater than about 0.1 milligrams, such as greater than about 0.5 milligrams, such as greater than about 1 milligram, such as greater than about 2 milligrams, such as greater than about 2.5 milligrams, such as greater than about 5 milligrams, such as greater than about 10 milligrams, such as greater than about 15 milligrams, such as greater than about 20 milligrams, such as greater than about 25 milligrams, such as greater than about 30 milligrams, such as greater than about 35 milligrams, such as greater than about 40 milligrams. In one embodiment, greater amounts of the Vitamin B compound or the derivative, or precursor thereof is present in each dosage vessel. For instance, the Vitamin B compound or the derivative, or precursor thereof can be present in each dosage vessel in an amount greater than about 50 milligrams, such as greater than about 60 milligrams, such as greater than about 80 milligrams, such as greater than about 100 milligrams, such as greater than about 200 milligrams, such as greater than about 300 milligrams, such as greater than about 400 milligrams, such as greater than about 500 milligrams, such as greater than about 600 milligrams, such as greater than about 700 milligrams, such as greater than about 800 milligrams, such as greater than about 900 milligrams, such as greater than about 1,000 milligrams, such as greater than about 1,200 milligrams, such as greater than about 1,400 milligrams, such as greater than about 1,600 milligrams, such as greater than about 1,800 milligrams, such as greater than about 2,000 milligrams, such as greater than about 2,500 milligrams, such as greater than about 3,000 milligrams, such as greater than about 3,500 milligrams, such as greater than about 4,000 milligrams, such as greater than about 4,500 milligrams, such as greater than about 5,000 milligrams, such as greater than about 5,500 milligrams, such as greater than about 6,000 milligrams. The Vitamin B compound or derivative, or precursor thereof is generally present in an amount less than about 10,000 milligrams, such as less than about 8,000 milligrams, such as less than about 7,000 milligrams.

In one embodiment, each dosage vessel can contain niacin generally in an amount from about 0.5 milligrams to about 200 milligrams, such as from about 1 milligram to about 100 milligrams. In another embodiment, each dosage vessel may contain niacinamide in an amount from about 100 milligrams to about 5,000 milligrams, such as from about 200 milligrams to about 2,000 milligrams. In still another embodiment, each dosage vessel can contain tryptophan in an amount from about 10 milligrams to about 10,000 milligrams, such as from about 60 milligrams to about 6,000 milligrams. In yet another embodiment, each dosage vessel can contain nicotinamide dinucleotide in an amount from about 10 milligrams to about 200 milligrams, such as in an amount from about 80 milligrams to about 20 milligrams.

As described above, in one embodiment, the composition of the present disclosure can be formulated as a nutritional supplement that is taken orally. It should be understood, however, that the composition of the present disclosure can be administered to a human or animal using any suitable method. In one embodiment, the composition can be incorporated into a topical composition that is intended to be applied to the skin of a user. When formulated as a topical composition, for instance, the collagen source combined with the Vitamin B compound, or derivative, or precursor thereof, can be blended with various ingredients and components. For instance, when formulated as a topical composition, the composition of the present disclosure can be blended with solvents, surfactants, emulsifiers, consistency factors, conditioners, emollients, skin caring ingredients, moisturizers, thickeners, lubricants, preservatives, and various different dermatological ingredients. When applied to the skin of a user, the composition of the present disclosure can be used to improve skin health or can be formulated so as to be absorbed into the body.

In addition to being directed to a composition, such as a nutritional supplement, the present disclosure is also directed to a method for administering the nutritional supplement to a human or animal. For example, in one embodiment, the present disclosure is directed to a method for treating joint pain including arthritis by administering to a mammal a nutritional supplement containing undenatured Type II collagen in combination with a Vitamin B compound, or derivative, or precursor thereof, or a GPR109a ligand. The nutritional supplement is administered to the mammal in a therapeutically effective amount so as to reduce joint pain or to otherwise treat join pain. In one embodiment, for instance, the reduction in joint pain is evidenced by improvements in range motion, an increase in levels of activity, or any activity that depends on having functional knees.

In other embodiments, the present disclosure is directed to a method of treating a mammal in order to improve immune health, in order to decrease cholesterol levels in the body, to reduce inflammation, or both. The method includes administering to the mammal the nutritional supplement as described above in a therapeutically effective amount sufficient to have one of the above effects.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A composition comprising:
a Type II collagen source combined with a Vitamin B compound, or derivative, or precursor thereof or a GPR109a receptor ligand.

2. A composition as defined in claim 1, wherein the composition comprises a nutritional supplement for taking orally.

3. A composition as defined in claim 1, wherein the composition comprises a topical composition for applying to skin.

4. A composition as defined in claim 1, wherein the Type II collagen source comprises an undenatured Type II collagen.

5. A composition as defined in claim 2, wherein the nutritional supplement is in the form of individual dosage vessels.

6. A composition as defined in claim 5, wherein the dosage vessels comprise tablets, capsules, gummy chewables, edible films, liquid suspensions, powders, syrups, or lozenges.

7. A composition as defined in claim 1, wherein the Vitamin B compound or derivative or precursor thereof or GPR109a receptor ligand comprises a nicotinate.

8. A composition as defined in claim 1, wherein the Vitamin B compound or derivative or precursor thereof or GPR109a receptor ligand comprises niacin.

9. A composition as defined in claim 1, wherein the Vitamin B compound or derivative or precursor thereof or GPR109a receptor ligand comprises niacinamide.

10. A composition as defined in claim 1, wherein the Vitamin B compound, or derivative, or precursor thereof or GPR109a receptor ligand comprises tryptophan.

11. A composition as defined in claim 1, wherein the Vitamin B compound or derivative or precursor thereof or GPR109a receptor ligand comprises nicotinamide dinucleotide.

12. A composition as defined in claim 1, wherein the Vitamin B compound or derivative, or precursor thereof or GPR109a receptor ligand comprises a niacinamide N-oxide, a salt of niacinamide, nicotinamide adenine dinucleotide phosphate, a tryptophan dipeptide, a nicotinyl alcohol, a nicotinic acid ester, a nicotinyl amino acid, a nicotinyl alcohol ester, or mixtures thereof.

13. A composition as defined in claim 5, wherein the individual dosage vessels contain from about 1 mg to about 15,000 mg of the Type II collagen.

14. A composition as defined in claim 5, wherein the individual dosage vessels contain from about 0.1 milligrams to about 10,000 milligrams of the Vitamin B compound or derivative, or precursor thereof or GPR109a receptor ligand.

15. A composition as defined in claim 13, wherein the Vitamin B compound or derivative or precursor thereof or GPR109a receptor ligand comprises niacin or nicotinamide dinucleotide and wherein the niacin or nicotinamide dinucleotide are present in each dosage vessel in an amount from about 0.1 milligrams to about 200 milligrams.

16. A composition as defined in claim 13, wherein the Vitamin B compound or derivative, or precursor thereof or GPR109a receptor ligand comprises niacinamide or tryptophan and the niacinamide or tryptophan are present in each dosage vessel in an amount from about 10 milligrams to about 8,000 milligrams.

17. A composition as defined in claim 1, wherein the Vitamin B compound or derivative, or precursor thereof or GPR109a receptor ligand comprises the GPR109a receptor ligand.

18. A composition as defined in claim 17, wherein the GPR109a receptor ligand comprises a fatty acid or salt thereof.

19. A composition as defined in claim 17, wherein the GPR109a receptor ligand comprises butyric acid or a butyrate.

20. A composition as defined in claim 17, wherein the GPR109a receptor ligand is derived from probiotic or dietary fiber metabolism.

21. A method of treating joint pain in a mammal comprising administering a composition to the mammal comprising a Type II collagen combined with a Vitamin B compound or derivative, or precursor thereof, the composition being administered to the mammal in a therapeutically effective amount sufficient to reduce joint pain in both healthy individuals and individuals with arthritis.

22. A method for reducing inflammation, improving immune system health, improving gastrointestinal health, improving brain health and/or lowering cholesterol in a mammal comprising administering to the mammal a composition comprising a Type II collagen source combined with a Vitamin B compound or derivative, or precursor thereof, the composition being administered to the mammal in a therapeutically effective amount sufficient to reduce inflammation, improve immune system, gastrointestinal and brain health, and/or lower cholesterol.
